(19) United States
(12) Patent Application Publication
(10) Pub. No.: US 2026/0165928 A1
(43) Pub. Date: Jun. 18, 2026

(54) Personal Care Compositions

(71) Applicant: Colgate-Palmolive Company, New York, NY (US)

(72) Inventors: Aaron COHEN, New Brunswick, NJ (US); Junhong MAO, Plainsboro, NJ (US); Baran TEOMAN, Lake Hiawatha, NJ (US); Tatiana BRINZARI, Franklin Park, NJ (US); Ningwei LI, North Brunswick, NJ (US); Ewelina LESNIAK, North Brunswick, NJ (US); Savitha RAJAGOPAL, Chandler, AZ (US); Dannielle Caswell AMAVISCA, Phoenix, AZ (US)

(73) Assignee: Colgate-Palmolive Company, New York, NY (US)

(21) Appl. No.: 19/409,870

(22) Filed: Dec. 5, 2025

Related U.S. Application Data
(60) Provisional application No. 63/735,480, filed on Dec. 18, 2024.

(51) Int. Cl. A61K 8/368; A61K 8/34; A61K 8/365; A61K 8/60; A61K 8/64; A61K 8/73; A61K 8/9789; A61Q 15/00; A61Q 19/08; A61Q 19/10

(52) U.S. Cl. CPC A61K 8/368; A61K 8/34; A61K 8/347; A61K 8/365; A61K 8/606; A61K 8/64; A61K 8/736; A61K 8/9789; A61Q 15/00; A61Q 19/08; A61Q 19/10; A61K 2800/30; A61K 2800/48

(57) ABSTRACT

Described herein are personal care compositions comprising: lactic acid; glycolic acid; salicylic acid; a cosmetically acceptable carrier, and one or more naturally derived thickening agents. The personal care composition has a low pH (about 1.0-2.0). Methods of making and using these compositions are also described.

### BACKGROUND

Skincare has many benefits besides recuperating a youthful and glowing appearance. Skin exfoliates dead skin cells naturally every day, however as a result of the aging process, sun damage or exposure to environmental pollutants, the natural peeling process slows and may stop altogether. Our skin becomes dull, dry, flaky and wrinkled. By gently exfoliating the buildup of dead skin, the firmness, the beauty and the even-toned skin are restored.

Many personal care products currently available to consumers are directed primarily to improving the health and/or appearance of skin. For example, there are a variety of topical skin care products available that are directed to delaying, minimizing, or even eliminating skin dryness, skin wrinkling, and other histological changes commonly associated with the aging of skin or environmental damage to human skin. As a result, the sale of personal care products has become a booming business in youth-conscious societies.

There is a growing need for personal care formulations with anti-aging exfoliating and brightening effects, which bring out a healthy looking radiance while evening skin tone. There is an additional need for personal care products that provide these benefits without including certain ingredients.

### BRIEF SUMMARY

Aspects of the invention are directed to personal care formulations and, particularly, to personal care formulations for controlled wounding, by exfoliating the stratum corneum/epidermis of the skin. When the stratum corneum is roughened by internal and external factors, the activation and regeneration of cells underneath the stratum corneum are increased. When keratin peeling is promoted, the skin becomes transparent and soft. The present personal care formulations will not leave any visible scars after their application. In some embodiments, the personal care formulations of the invention are referred to as exfoliating formulations and/or chemical peels. They are meant to be applied to the human subject in a professional environment. After treatment, the human subject is expected to have a younger looking, smoother, more even-toned skin with a glowing appearance. The personal care formulations of the invention exfoliate the skin, making it smooth and soft, and also improve problems of skin peeling caused by dryness and aging, fortifying the skin barrier.

The personal care formulations of the invention are intended for dermatological topical application and they deliver very low stinging or pain sensation, upon application. The criteria of these personal care formulations are achieved through a combination of alpha hydroxy acids (AHAs), beta hydroxy acids (BHAs), and polyhydroxy acids (PHAs). The criteria of these personal care formulations are also achieved through a combination of alpha hydroxy acids (AHAs), and beta hydroxy acids (BHAs). In addition, the personal care formulations include materials that mitigate stinging, pain, are anti-inflammatory and soothing.

The personal care formulations of the invention were evaluated by determining their effect on the surface of the skin. The smoothness of the skin surface was evaluated after treatment with personal care formulations of the invention. The smoothness of the skin was determined with microscopic and nanoscopic techniques, specifically light microscopy (LM) and atomic force microscopy (AFM).

In accordance with an aspect of the invention, provided is a personal care formulation comprising an alpha hydroxy acid; a beta hydroxy acid; and optionally a polyhydroxy acid comprising gluconodeltalactone. In accordance with an aspect of the invention, provided is a personal care formulation comprising alpha hydroxy acids and a beta hydroxy acid.

According to another aspect of the invention, a personal care formulation is provided that comprises: lactic acid; glycolic acid; salicylic acid; optionally gluconolactone; and a cosmetically acceptable carrier.

Some embodiments of the present invention provide a personal care formulation that comprises from about from about 7 wt. % to about 12 wt. % lactic acid; from about 8 wt. % to about 40 wt. % glycolic acid; from about 1 wt. % to about 8 wt. % salicylic acid; a naturally derived thickening agent; and a cosmetically acceptable carrier; wherein the personal care composition is substantially free of trichloroacetic acid (TCA). Preferably, the composition is free of TCA.

### BRIEF DESCRIPTION OF THE DRAWINGS

The present invention will become more fully understood from the detailed description and the accompanying drawings, wherein:

FIG. 1 is a graph of the viscosity results comparing formulas comprising either 10% glycolic acid or 30% glycolic acid, with or without 4% chitosan in water.

FIG. 2 is a graph of the amount of penetrated glycolic acid from formulations comprising 10% glycolic acid with or without 4% chitosan, measured within the skin over time.

FIG. 3 is a graph of the amount of glycolic acid from formulations comprising 10% glycolic acid with or without 4% chitosan, measured as penetrating through the skin and into the receiver fluid, over time.

FIG. 4 is a graph of the amount of glycolic acid from formulations comprising 10% glycolic acid with or without 4% chitosan, measured from different locations (swabs, tape stips, skin remainder, and receiver fluid) at a single time point of 3 hours.

### DETAILED DESCRIPTION

It is to be understood that the foregoing detailed description of the invention and embodiments, are exemplary and explanatory only and are not restrictive in any way of the claimed invention. As used herein, the use of the plural tense includes the singular tense unless specifically stated otherwise. Similarly, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2.0 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the personal care formulations of the instant disclosure can be free or essentially free of all components and elements positively recited throughout the instant disclosure. In some instances, the personal care formulation of the present invention may be substantially free of non-incidental amounts of the ingredient(s) or compound(s) described herein. A non-incidental amount of an ingredient or compound is the amount of that ingredient or compound that is added into the personal care formulation by itself. For example, a personal care formulation may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care formulation includes both components (or the formulation includes more than two components that overlap), an overlapping compound does not represent more than one component. For example, certain compounds (e.g., ethanol) may be characterized as both a penetration agent and a carrier. If a particular personal care formulation includes both a penetration agent and a carrier, ethanol will serve only as either a penetration agent or a carrier—not both.

As used herein, reference to a compound comprising several isomers or stereoisomers includes all the isomeric or stereoisomeric forms of that compound. For example when reference is made to lactic acid, it can refer to 1-(+)-lactic acid or d-(−)-lactic acid, or to a mixture thereof. Unless stated otherwise, all percentages of the personal care formulations ingredients, given in this specification, are by weight based on the total personal care formulation weight of 100%.

As used herein, reference to an acid, may include all chemical salts and/or esters of that acid. For example when reference is made to lactic acid, it can refer to a lactate salt or to a lactate ester. Some of the personal care formulations of the invention may comprise ingredients which have several properties, for instance some of the anti-inflammatory ingredients may have soothing properties and/or wound healing properties as well as antipollution properties and antioxidation properties.

As used herein, the term “exfoliating” refers to a cosmetic technique which improves the skin appearance by removing or facilitating the removal of dead skin cells which accumulate on the topmost layer of skin and/or stratum corneum enabling the new layer of skin cells to come to the surface and grow. Exfoliating formulations may comprise gritty solids of varying particle size which provide sensory signals of cleansing when rubbed into the skin. The abrasive nature of these particles suspended in a suitable base provides a smooth after-feel. In some embodiments, the personal care formulations of the present invention are free from gritty solids. In some embodiments, the personal care formulations of the present invention are free of particles with abrasive properties.

As used herein, the term “chemical peel” refers to a skin exfoliant with a low pH from about 0.05 to about 4. Chemical peels are caused by agents that interact with the complex structure of the skin, removing the outer layer to expose the underlying layers. In general, chemical peels dissolve dead cells and may have a watery, gel-like consistency. Chemical peels may comprise fruit enzymes, fruit acids, and/or metal complexes. In some embodiments, however, the personal care formulations of the present invention are free of fruit enzymes, fruit acids, and/or metal complexes. Although chemical peels may comprise retinol, in some embodiments the personal care formulations of the inventions are free from retinol. In some embodiments, the personal care formulations of the present invention comprise bakuchiol.

The personal care formulations of the invention may be applied on the skin of the human subject. In general, the personal care formulations of the invention may be applied to the face, the back of the hands, the knees, the heels or on the skin of any other parts of the human subject. In some preferable embodiments, the personal care formulations of the invention may be applied to the face.

As used herein, the term “topical application” refers to applying or spreading the personal care formulations of the invention on the surface of the skin of the human subject.

As used herein the term “dermatological” refers to skin.

As used herein the term “smoothness of the skin” refers to skin which lacks roughness, without bends and irregularities, and silky to the touch.

As used herein, the terms “surface smoothness” and “surface roughness” refer to the surface texture; they were quantified by the deviations in the direction of the normal vector of a real surface from its ideal form. If these deviations are large, the surface is rough, if they are small, the surface is smooth.

Surface smoothness and surface roughness may be measured with a skidded gage, as in methods including, but not limited to, interferometric optical profilometry or stylus profilometry. The skin surface roughness in the present invention was determined using Atomic Force Microscopy (AFM).

Atomic Force Microscopy is a three-dimensional scanning technique that has <0.2 nm spatial resolution and the ability to measure most types of materials. Surface roughness acquisition via AFM was obtained through the use of a cantilever with a sharp tip at its end that was used to scan the surface. As the tip contacts the surface, the cantilever bends, and the bending is detected using a laser diode and a split photodetector. This bending is indicative of the tip-sample interaction force. When the tip was brought into proximity of a sample surface, forces between the tip and the sample lead to a deflection of the cantilever according to Hooke's law. Surface roughness may be measured in terms of a number of parameters known in the art.

Aspects of the invention are directed to personal care formulation formulated to improve the appearance of skin and/or skin health. In one embodiment, the personal care formulation is a dermatological chemical peel and/or dermatological exfoliating formulation. The inventors discovered that certain combinations of alpha hydroxy acids, beta hydroxy acid, and optionally polyhydroxy acid in certain weight ratios provide enhanced benefits to the skin.

In accordance with one aspect of the invention, provided is a personal care formulation comprising one or more alpha hydroxy acids, and a beta hydroxy acid, and optionally polyhydroxy acid. According to another aspect of the invention, a personal care formulation is provided that comprises one or more alpha hydroxy acids; and a beta hydroxy acid; wherein the personal care formulation has a weight ratio of alpha hydroxy acid to beta hydroxy acid of about 20:1 to about 1:20, or about 19:1. In one embodiment, the present invention provides personal care formulations comprising a plurality of ingredients selected from penetration agent(s), alpha hydroxy acid(s), beta hydroxy acid(s), thickening agent(s), humectants/hydrator(s), soothing agent(s), carrier(s), antioxidant(s), anti-inflammation agent(s), wound healing agent(s), pH adjuster(s), amino acid(s), anti-aging agent(s), and a combination of two or more thereof.

In some preferred embodiments, the personal care composition is formulated to have a weight ratio of alpha hydroxy acid to beta hydroxy acid of about 20:1 to about 1:20. For example, the weight ratio of the alpha hydroxy acid to polyhydroxy acid may be about 20:1 to about 1:20, about 19:1 to about 1:19, about 18:1 to about 1:18, about 15:1 to about 1:15; from about 13:1 to about 1:13, about 12:1 to about 1:12, or about 10:1 to about 1:10. Preferably, the personal care composition is formulated to have a weight ratio of alpha hydroxy acid to beta hydroxy acid of 19:1.

The personal care formulation may comprise one or more penetration agent(s). In general, penetration agents may include, but are not limited to, monoalcohols, ethanol, witch hazel, urea, fatty acids, glycols or combinations thereof. In some embodiments, the penetration agents include monoalcohols, ethanol, propanol, butanol, witch hazel, glycols or combinations thereof. For instance, the penetration agents may include monoalcohol, anhydrous ethyl alcohol, water, and witch hazel. In at least one embodiment, the penetration agents are ethanol and witch hazel.

The personal care formulation may comprise one or more penetration agent(s) in an amount from about 5 by weight to about 99 wt. %, or from about 25 wt. % to about 70 wt. %, or from about 45 to about 55 wt. %, relative to the total weight of the personal care formulation. In some cases, the personal care formulation includes one or more penetration agent(s) in an amount from about 5 to about 90 wt. %, about 5 to about 75 wt. %, about 5 to about 50 wt. %, about 5 to about 25 wt. %, about 5 to about 15 wt. %; from about 25 to about 90 wt. %, about 25 to about 75 wt. %, about 25 to about 50 wt. %, about 25 to about 40 wt. %; from about 40 to about 90 wt. %, about 40 to about 75 wt. %, about 40 to about 60 wt. %; from about 60 to about 90 wt. %, about 60 to about 80 wt. %, about 60 to about 70 wt. %; from about 75 to about 90 wt. %, about 75 to about 85 wt. %; from about 80 to about 90 wt. %, about 85 to about 90 wt. %, or any ranges and subranges thereof, based on the total weight of the personal care formulation. In at least one embodiment, the personal care formulation comprises penetration agent(s) in an amount from about 45 wt. % to about 55 wt. %, based on the total weight of the personal care formulation.

The personal care formulation can include one or more alpha hydroxy acid(s) and/or a salt thereof. For example, the personal care formulation may include one or more one or more alpha hydroxy acid(s), such as those selected from C₂ to C₇ alpha-hydroxy acid or C₄ to C₆ alpha-hydroxy acid. In some embodiments, the personal care formulation includes a salt of an alpha-hydroxy acid. The salt of the alpha-hydroxy acid is preferably a sodium salt or a potassium salt. In at least one embodiment, the salt is a sodium salt (i.e., the cation associated with the salt is a sodium). Non-limiting examples of alpha hydroxy acids include but are not limited to, mandelic acid, glycolic acid, citric acid, lactic acid, malic acid, tartaric acid, phytic acid, hydroxycaprylic acid, hydroxycapric acid, glutaric acid, gluconic acid, or a combination of two or more thereof. In some embodiments, the alpha hydroxy acids are citric acid, mandelic acid, glycolic acid, lactic acid or a combination of two or more thereof. In a certain instance, the alpha-hydroxy acid is selected from malic acid, tartaric acid, alpha-hydroxy glutaric acid, gluconic acid, a salt thereof, and a combination of two or more thereof. Yet in further instances, the alpha-hydroxy acid or salt thereof is selected from lactic acid, malic acid, and sodium-D-gluconate. In at least one embodiment, the alpha hydroxy acid is lactic acid and glycolic acid.

The personal care formulations of the invention may comprise alpha hydroxy acids in an amount from about 8 to about 48 wt. % or from about 10 to about 44 wt. % relative to the total weight of the personal care formulation. For example, the amount of alpha hydroxy acids in the personal are formulations may be from about 10 to about 44 wt. %, about 12 to about 44 wt. %, about 14 to about 44 wt. %, about 16 to about 44 wt. %, about 18 to about 44 wt. %, about 20 to about 44 wt. %; from about 10 to about 40 wt. %, about 12 to about 40 wt. %, about 14 to about 40 wt. %, about 16 to about 40 wt. %, about 18 to about 40 wt. %, about 20 to about 40 wt. %, about 22 to about 40 wt. %; from about 8 to about 38 wt. %, about 10 to about 38 wt. %, about 12 to about 38 wt. %, about 14 to about 38 wt. %, about 16 to about 38 wt. %, about 18 to about 38 wt. %, about 20 to about 38 wt. %; from about 22 to about 38 wt. %, about 24 to about 38 wt. %, about 26 to about 38 wt. %, about 28 to about 38 wt. %, about 30 to about 38 wt. %, about 32 to about 38 wt. %, about 34 to about 38 wt. %, or about 36 to about 38 wt. %, including any range or subrange thereof, based on the total weight of the personal care formulation. In at least one embodiment, the personal care formulation comprises alpha hydroxy acids in an amount of about 38 wt. %, based on the total weight of the personal care formulation.

The personal care formulation typically comprises one or more beta hydroxy acid(s). Non-limiting examples of beta hydroxy acids include salicylic acid, propionic acid, beta-hydroybutyric acid, beta-hydroxy beata-methylbutyric acid, carnitine, and combinations of two or more thereof. The beta hydroxy acids may in some cases be selected from salicylic acid, esters of salicylic acid, sodium salicylate, beta hydroxybutanoic acid, tropic acid, trethocanic acid, beta hydroxyl acids obtained from white willow bark extract and/or wintergreen leaves, and combinations of two or more thereof. In some embodiments, the beta hydroxy acid(s) comprises salicylic acid, sodium salicylate, beta hydroxyl acids of willow bark extract or combinations thereof. In one embodiment, the beta hydroxy acid is salicylic acid.

The personal care formulations of the invention may comprise beta hydroxy acids in an amount from about 0.5 to about 10 wt. %, or from about 1.5 to about 3 wt. %, relative to the total weight formulation. For example, the amount of beta hydroxy acids present in the personal care formulation may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 10 wt. %, about 2 to about 8 wt. %, about 2 to about 6 wt. %, about 2 to about 5 wt. %, about 2 to about 4 wt. %, about 2 to about 3 wt. %; about 3 to about 10 wt. %, about 3 to about 8 wt. %, about 3 to about 6 wt. %, about 3 to about 5 wt. %, about 3 to about 4 wt. %, including ranges and subranges thereof, based on the total weight of the personal care formulation. In one instance, the personal care formulation comprises beta hydroxy acids from about 1.5 to about 2 wt. %, or about 2 wt. %, based on the total weight of the personal care formulation.

The personal care formulations may optionally include one or more polyhydroxy acid(s). The polyhydroxy acids may include, but are not limited to, gluconolactone, gluconic acid, galactose, lactobionic acid, or combinations thereof. In some embodiments, the polyhydroxy acids are gluconolactone, lactobionic acid or combinations thereof. Preferably, the polyhydroxy acid is glucanodeltalactone. Glucanodeltalactone is also referred to as glucono-1,5-lactone or GDL.

The personal care formulations of the invention may comprise polyhydroxy acid(s) in an amount from about 2 to about 20 wt. %, about 7 to about 20 wt. %, or from about 10% to about 15 wt. % by weight, relative to the total weight of the personal care formulation. For example, the polyhydroxy acid(s) may be present in the personal care formulation in an amount from about 2 to about 18 wt. %, about 2 to about 16 wt. %, about 2 to about 14 wt. %, about 2 to about 12 wt. %, about 2 to about 10 wt. %, about 2 to about 8 wt. %, about 2 to about 6 wt. %, about 2 to about 4 wt. %; from about 5 to about 20 wt. %, about 5 to about 18 wt. %, about 5 to about 16 wt. %, about 5 to about 14 wt. %, about 5 to about 12 wt. %, about 5 to about 10 wt. %, about 5 to about 8 wt. %; from about 8 to about 20 wt. %, about 8 to about 18 wt. %, about 8 to about 16 wt. %, about 8 to about 14 wt. %, about 8 to about 12 wt. %, about 8 to about 10 wt. %; from about 10 to about 20 wt. %, about 10 to about 18 wt. %, about 10 to about 16 wt. %, about 10 to about 14 wt. %, about 10 to about 12 wt. %; from about 12 to about 20 wt. %, about 12 to about 18 wt. %, about 12 to about 16 wt. %, about 12 to about 14 wt. %; from about 14 to about 20 wt. %, about 14 to about 18 wt. %, about 14 to about 16 wt. %; from about 16 to about 20 wt. %, about 16 to about 18 wt. %, including any range or subrange thereof, based on the total weight of the personal care formulation. In at least one instance, the personal care formulation comprises polyhydroxy acid(s) from about 10 to about 15 wt. %, based on the total weight of the personal care formulation.

In general, humectants include, but are not limited to, polyhydric alcohols such as glycerin, butylene glycol, 1,3-propanediol, sorbitol, xylitol or low molecular weight polyethylene glycols (PEGs), polyoxyethylenes or combinations thereof. In some embodiments, the humectant agents are sorbitol (preferably, non-crystal sorbitol), butylene glycol,1,3-propanediol, glycerin (e.g., vegetable refined glycerin), polyoxyethylene glycol or combinations thereof. In at least one embodiment, the humectants are chosen from glycerin, butylene glycol and 1,3-propanediol. Typically, the humectant is vegetable refined glycerin. Other suitable humectants include, but are not limited to, polyglutamic acid, saccharide isomerate or combinations thereof. In at least one instance, the humectant is saccharide isomerate. Saccharide isomerate can be commercially obtained from, e.g., DSM under the trademark of Pentavitin™.

The personal care formulation of the invention may comprise humectants in an amount from about 1 to about 20 wt. %, or from about 5 to about 10 wt. %, or from about 10 to about 15 wt. %, relative to the total weight of the personal care formulation. For example, the humectants may be present in the personal care formulation in an amount from about 1 to about 18 wt. %, about 1 to about 16 wt. %, about 1 to about 14 wt. %, about 1 to about 12 wt. %, about 1 to about 10 wt. %, about 1 to about 8 wt. %, about 1 to about 6 wt. %, about 1 to about 4 wt. %; from about 2 to about 20 wt. %, about 2 to about 18 wt. %, about 2 to about 16 wt. %, about 2 to about 14 wt. %, about 2 to about 12 wt. %, about 2 to about 10 wt. %, about 2 to about 8 wt. %, about 2 to about 6 wt. %, about 2 to about 4 wt. %; from about 5 to about 20 wt. %, about 5 to about 18 wt. %, about 5 to about 16 wt. %, about 5 to about 14 wt. %, about 5 to about 12 wt. %, about 5 to about 10 wt. %, about 5 to about 8 wt. %; from about 8 to about 20 wt. %, about 8 to about 18 wt. %, about 8 to about 16 wt. %, about 8 to about 14 wt. %, about 8 to about 12 wt. %, about 8 to about 10 wt. %; from about 10 to about 20 wt. %, about 10 to about 18 wt. %, about 10 to about 16 wt. %, about 10 to about 14 wt. %, about 10 to about 12 wt. %; from about 12 to about 20 wt. %, about 12 to about 18 wt. %, about 12 to about 16 wt. %, about 12 to about 14 wt. %; from about 14 to about 20 wt. %, about 14 to about 18 wt. %, about 14 to about 16 wt. %; from about 16 to about 20 wt. %, about 16 to about 18 wt. %, including any range or subrange thereof, based on the total weight of the personal care formulation. In at least one embodiment, the personal care formulation comprises humectant(s) in an amount from about 1 to about 5 wt. %, based on the total weight of the personal care formulation.

In general, soothing agents include, but are not limited to, natural extracts comprising: Centella asiatica extract, Polygonum cuspidatum root extract, Scutellaria baicalensis root extract, Camellia sinensis leaf extract, Glycyrrhiza glabra (licorice) root extract, Chamomilla recutita (matricaria) flower extract, Rosmarinus officinalis (rosemary) leaf extract, tea (Camellia sinensis) leaf extract, Poria cocos extract, Phragmites karka extract, lidocaine, chia seed extract, Rosa centifolia flower extract, Aloe vera, allantoin, D-panthenol, turmeric, avocado oil, and lichen extract, or a combination of two or more thereof. Some soothing agents are sold under the names of MultiEx BSASM™, SyriCalm CLR™, and ENFIBIN™ (Butylene Glycol (and) Acetyl Tripeptide-74 Amide, sold by SupadElixir).

The personal care formulation of the invention may, optionally, comprise soothing agent(s) in an amount from about 0 to about 5 wt. %, or from about 0.5 to about 3 wt. %, relative to the total weight of the personal care formulation. For instance, the amount of soothing agent(s) present in the personal are formulation may be from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care formulation.

The personal care formulation may include one or more carrier(s). In general, carriers include but are not limited to, water or alcohols (e.g., ethanol, isopropanol or mixtures thereof). In at least one embodiment, the carrier comprises water. The amount of carrier present in the personal care formulation may vary, e.g., from about 5 to about 90 wt. %, based on the total weight of the personal care formulation. In some cases, the personal care formulation includes one or more carrier(s) in an amount from about 5 to about 75 wt. %, about 5 to about 50 wt. %, about 5 to about 25 wt. %, about 5 to about 15 wt. %; from about 25 to about 90 wt. %, about 25 to about 75 wt. %, about 25 to about 50 wt. %, about 25 to about 40 wt. %; from about 40 to about 90 wt. %, about 40 to about 75 wt. %, about 40 to about 60 wt. %; from about 60 to about 90 wt. %, about 60 to about 80 wt. %, about 60 to about 70 wt. %; from about 75 to about 90 wt. %, about 75 to about 85 wt. %; from about 80 to about 90 wt. %, about 85 to about 90 wt. %, or any ranges and subranges thereof, based on the total weight of the personal care formulation.

In some embodiments, the personal care formulation comprises one or more antioxidant(s). Examples of antioxidants include, but are not limited to, Camelliajaponica extract, quercetin or combinations thereof. Quercetin is commercially available under the trademark of Quercevita™ Typically the antioxidant is Camelliajaponica extract. Camelliajaponica extract is sold under the name of RedSnow®. The personal care formulation of the invention may comprise one or more antioxidant(s) from about 0 to about 5 wt. %, or from about 0.5 to about 3 wt. %, relative to the total weight of the personal care formulation. In some cases, the amount of antioxidant(s) present in the personal are formulation may be from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care formulation.

In general, wound healing agents include but are not limited to, triterpenes, triterpenoids, oleanolic acid, maslinic acid, asiaticoside or combinations thereof. In some embodiments, the wound healing agents include asiaticoside and oleanolic acid or a combination of two or more thereof. Typically, the wound healing agent is asiaticoside. Asiaticoside has antipollution, anti-inflammatory and/or antioxidation properties. The personal care formulation of the invention may comprise one or more wound healing agent(s) from about 0 to about 5 wt. %, or from about 0.5 to about 3 wt. %, relative to the total weight of the personal care formulation. In some cases, the amount of wound healing agent (s) present in the personal are formulation may be from about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 5 wt. %, about 0.5 to about 4 wt. %, about 0.5 to about 3 wt. %, about 0.5 to about 2 wt. %, about 0.5 to about 1 wt. %; from about 1 to about 5 wt. %, about 1 to about 4 wt. %, about 1 to about 3 wt. %, about 1 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care formulation.

The personal care formulation in some instances comprises one or more amino acid(s). In general, amino acids include, but are not limited to, arginine, methionine, taurine, leucine, glycine, valine, lysine, alanine, cysteine, histidine, leucine, proline, serine, tyrosine or a combination of two or more thereof. In some embodiments, the amino acids include arginine, taurine, glycine, histidine, methionine, lysine, proline, leucine or combinations thereof. Typically, the amino acid is arginine. The personal care formulations of the invention may comprise amino acids from about 0.5 by weight to about 10 wt. %, or from about to 1 to about 2 wt. %, or from about 2 to about 4 wt. %, or from about 4 to about 6 wt. %, or from about 6 to about 8 wt. %, or from about 8 to about 10 wt. %, relative to the total weight of the personal care formulation. In some embodiments, the personal care formulation comprises one or more amino acid in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 4 to about 10 wt. %, about 4 to about 8 wt. %, about 4 to about 6 wt. %; from about 6 to about 10 wt. %, about 6 to about 8 wt. %; from about 8 to about 10 wt. %, or any range or subrange thereof, based on the total weight of the personal care formulation. In at least one embodiment, the personal care formulations of the invention comprise wound healing agents from about 1 to about 2 wt. %, based on the total weight of the personal care composition.

The personal care formulation may comprise one or more anti-aging agent(s). In general, anti-aging agents may include, but are not limited to, tripeptides acceptable for cosmetic, cosmeceutical, and over-the-counter drug products that can be delivered efficiently through the transdermal route. Examples of anti-aging agents include, but are not limited to, SYNDERMIN™ Solution 100 (Palmitoyl sh-Tripeptide-1 Amide), SYNEPIN™ Solution 100 (Palmitoyl sh-Tripeptide-3 Amide), ADIPONIN™ Solution 200 (Acetyl Tripeptide-54 Amide), WINHIBIN™ Solution 200 (Palmitoyl Tripeptide-53 Amide), BINTERIN™ Solution 400 (Palmitoyl sh-Tripeptide-4 Amide), ENFIBIN™ Solution 100 (Acetyl Tripeptide-74 Amide), HYDRIN™ Solution 400 (Acetyl Tripeptide-54 Amide (and) Palmitoyl Tripeptide-53 Amide), SOOTHIN™ Solution 500 (Palmitoyl sh-Tripeptide-4 Amide (and) Acetyl Tripeptide-74 Amide), CLARIN™ Solution 400 (Palmitoyl sh-Tripeptide-1 Amide (and) Palmitoyl Tripeptide-54 Amide (and) Palmitoyl Tripeptide-53 Amide), BRIGHTIN™ Solution 400 (Acetyl Tripeptide-53 Amide (and) Acetyl Tripeptide-54 Amide (and) Acetyl Tripeptide-71 Amide), and ANTIAGIN™ II Solution 300 (Palmitoyl sh-Tripeptide-3 Amide (and) Palmitoyl sh Tripeptide-1 Amide (and) Acetyl Tripeptide-54 Amide (and) Acetyl Tripeptide-74 Amide), all available from SupadElixir Co. Ltd. Additional anti-aging agents include QUORANYS (Glycerin (and) Morinda Citrifolia Extract), available from Barnet Products, and VOLUNAGE® (peony root (Paeonia albiflora) extract), available from Silab).

The personal care formulation in some instances comprises one or more thickening agents. In general, thickening agents include, but are not limited to, chitosan. The personal care formulations of the invention may comprise thickening agents from about 0.5 by weight to about 5 wt. %, or from about 1 to about 4 wt. %, or from about 2 to about 3 wt. %, or from about to 1 to about 2 wt. %, relative to the total weight of the personal care formulation. In some embodiments, the personal care formulation comprises one or more thickening agents in an amount from about 0.5 to about 4 wt. %, about 0.5 to about 3 wt. %, about 0.5 to about 2 wt. %, about 0.5 to about 1 wt. %; about 1 to about 3 wt. %, about 1 to about 2 wt. %; about 2 to about 5 wt. %, about 2 to about 4 wt. %; or any range or subrange thereof, based on the total weight of the personal care formulation. In at least one embodiment, the personal care formulations of the invention comprise thickening agents from about 1 to about 2 wt. %, based on the total weight of the personal care composition.

In one embodiment, the present invention provides a personal care formulation which is an exfoliating formulation, comprising penetration agents, alpha hydroxy acids, beta hydroxy acids, polyhydroxy acids, humectants, soothing agents, anti-inflammatory, carriers, pH adjusters, antioxidants, wound healing agents, amino acids or combinations thereof. In one embodiment, the present invention provides a personal care formulation, which is a chemical peel, and comprises one or more of penetration agents, alpha hydroxy acids, beta hydroxy acids, polyhydroxy acids, humectants, soothing agents, anti-inflammatory, carriers, pH adjusters, antioxidants, wound healing agents, amino acids, anti-aging agents, or combination of two or more thereof.

In one embodiment, the present invention provides a personal care formulation comprising: penetration agents, wherein the penetration agents include denatured alcohol, anhydrous ethyl alcohol, water, witch hazel, urea, fatty acids, glycols or combinations thereof; alpha hydroxy acids, wherein the alpha hydroxy acids include citric acid, mandelic acid, glycolic acid, lactic acid, malic acid, phytic acid, hydroxycaprylic acid, hydroxycapric acid, tartaric acid or combinations thereof, beta hydroxy acids, wherein the beta hydroxy acids include salicylic acid, esters of salicylic acid, sodium salicylate, white willow bark extract, wintergreen leaves, beta hydroxybutanoic acid, tropic acid, trethocanic acid or combinations thereof, polyhydroxy acids, wherein the polyhydroxy acids include gluconolactone, gluconic acid, galactose, lactobionic acid or combinations thereof, humectants, wherein the humectants include polyhydric alcohols such as vegetable refined glycerin, butylene glycol, 1,3-propanediol, non crystal sorbitol, xylitol or low molecular weight polyethylene glycols, polyoxyethylenes, polyglutamic acid, saccharide isomerate or combinations of two or more thereof, soothing agents, wherein the soothing agents, include natural extracts comprising: Centella asiatica extract, Polygonum cuspidatum root extract, Scutellaria baicalensis root extract, Camellia sinensis leaf extract, Glycyrrhiza glabra root extract, Chamomilla recutita flower extract, Rosmarinus officinalis leaf extract, tea leaf extract, Poria cocos extract, Phragmites karka extract or combinations thereof, antioxidants, wherein the antioxidants, include Camelliajaponica extract, quercetin or combinations thereof, wound healing agents, wherein the wound healing agents include triterpenes, triterpenoids, oleanolic acid, maslinic acid, asiaticoside or combinations thereof, amino acids, wherein the amino acids include arginine, methionine, taurine, leucine, glycine, valine, lysine, alanine, cysteine, histidine, leucine, proline, serine, tyrosine or combinations thereof.

In one embodiment, the present invention provides a personal care formulation comprising: penetration agents, wherein the penetration agents include denatured alcohol, anhydrous ethyl alcohol, witch hazel, or combinations thereof, alpha hydroxy acids, wherein the alpha hydroxy acids include citric acid, glycolic acid, lactic acid or combinations thereof, beta hydroxy acids, wherein the beta hydroxy acids include salicylic acid, sodium salicylate, willow extract, or combinations thereof, polyhydroxy acids, wherein the polyhydroxy acids include gluconolactone or lactobionic acid, or combinations thereof, humectants, wherein the humectants include non-crystal sorbitol, butylene glycol,1,3-propanediol, vegetable refined glycerin or polyoxyethylene glycol polyglutamic acid, saccharide isomerate, or combination thereof, soothing agents, wherein the soothing agents, include natural extracts comprising: Centella asiatica extract, Polygonum cuspidatum root extract, Scutellaria baicalensis root extract, Camellia sinensis leaf extract, Glycyrrhiza glabra root extract, Chamomilla recutita flower extract, Rosmarinus officinalis leaf extract, tea leaf extract, Poria cocos extract, Phragmites karka extract, or combinations thereof, antioxidants, wherein the antioxidants include Camellia japonica extract, quercetin or combinations thereof, wound healing agents, wherein the wound healing agents include asiaticoside, oleanolic acid, or combinations thereof, amino acids, wherein the amino acids include arginine, taurine, glycine, histidine, methionine, lysine, proline, leucine or combinations thereof.

In one embodiment, the present invention provides a personal care formulation comprising: penetration agents, wherein the penetration agents include anhydrous ethyl alcohol, and witch hazel; alpha hydroxy acids, wherein the alpha hydroxy acids include lactic acid; beta hydroxy acids, wherein the beta hydroxy acids include salicylic acid; polyhydroxy acids, wherein the polyhydroxy acids include gluconolactone; humectants, wherein the humectants include butylene glycol,1,3-propanediol and vegetable refined glycerin, saccharide isomerate; soothing agents, wherein the soothing agents, include natural extracts comprising: Centella asiatica extract, Polygonum cuspidatum root extract, Scutellaria baicalensis root extract, Camellia sinensis leaf extract, Glycyrrhiza glabra root extract, Chamomilla recutita flower extract, Rosmarinus officinalis leaf extract, tea leaf extract, Poria cocos extract, Phragmites karka extract, or combinations thereof; antioxidants, wherein the antioxidants include Camellia japonica extract, quercetin, or combinations thereof, wound healing agents, wherein the wound healing agents include asiaticoside; amino acids, wherein the amino acids include arginine. In one embodiment, the present invention provides a personal care formulation comprising: anhydrous ethyl alcohol, witch hazel, salicylic acid, butylene glycol,1,3-propanediol, vegetable refined glycerin, saccharide isomerate, gluconolactone, lactic acid, demineralized water, Centella asiatica extract, Polygonum cuspidatum root extract, Scutellaria baicalensis root extract, Camellia sinensis leaf extract, Glycyrrhiza glabra root extract, Chamomilla recutita flower extract, Rosmarinus officinalis leaf extract, tea leaf extract, Poria cocos extract, Phragmites karka, Camellia japonica extract, quercetin, asiaticoside, arginine, and a combination of two or more thereof.

In one embodiment, the present invention provides a personal care formulation comprising: anhydrous ethyl alcohol, witch hazel, salicylic acid, butylene glycol,1,3-propanediol, vegetable refined glycerin, saccharide isomerate, gluconolactone, lactic acid, demineralized water, Centella asiatica extract, Polygonum cuspidatum root extract, Scutellaria baicalensis root extract, Camellia sinensis leaf extract, Glycyrrhiza glabra root extract, Chamomilla recutita flower extract, Rosmarinus officinalis leaf extract, tea leaf extract, Poria cocos extract, Phragmites karka, Camelliajaponica extract, asiaticoside, and arginine.

In one embodiment, the present invention provides a personal care formulation comprising: anhydrous ethyl alcohol, witch hazel, salicylic acid, vegetable refined glycerin, saccharide isomerate, gluconolactone, lactic acid, demineralized water, asiaticoside and arginine. In one embodiment, the present invention provides a personal care formulation comprising: anhydrous ethyl alcohol, witch hazel, salicylic acid, vegetable refined glycerin, saccharide isomerate, gluconolactone, lactic acid, demineralized water, Centella asiatica extract, Polygonum cuspidatum root extract, Scutellaria baicalensis root extract, Camellia sinensis leaf extract, Glycyrrhiza glabra root extract, Chamomilla recutita flower extract, Rosmarinus officinalis leaf extract, tea leaf extract, Poria cocos extract, Phragmites karka, Camellia japonica extract, asiaticoside and arginine.

In one embodiment, the present invention provides a personal care formulation comprising: anhydrous ethyl alcohol, witch hazel, salicylic acid, vegetable refined glycerin, polyglutamic acid, gluconolactone, lactic acid, mandelic acid and demineralized water.

The pH of the personal care formulations is an important factor in the availability of the acid and the stability of the formulation. A low pH is necessary in order to enhance the penetration of the acid into the stratum corneum. Acids, bases, and buffers may be used to adjust the pH of the formulations of the invention. pH adjusters include but are not limited to, ammonia, sodium carbonate, sodium hydroxide, triethanolamine, hydrochloric acid, phosphoric acid, sodium hydrogen phosphate, sodium dihydrogen phosphate, citric acid, and the like. Typically, the personal care formulations of the invention comprise sodium hydroxide as pH adjuster. The personal care formulations of the present invention have a pH comprised from about 0.15 to about 3.50, or from about 0.25 to about 3.00, or from about 0.50 to about 2.50, or from about 0.60 to about 2.00, or from about 0.65 to about 1.75, or from about 0.75 to about 1.50, or from about 0.80 to about 1.25, or from about 0.90 to about 1.20, or from about 1.00 to about 1.15, or about 1.15.

In one embodiment, the present invention provides a personal care formulation comprising from about 7 wt. % to about 12 wt. % lactic acid; from about 8 wt. % to about 40 wt. % glycolic acid; from about 1 wt. % to about 8 wt. % salicylic acid; a naturally derived thickening agent; and a cosmetically acceptable carrier; wherein the personal care composition is substantially free of trichloroacetic acid (TCA).

In some embodiments, provided are personal care formulations having a composition comprising one or more components and/or ingredients in accordance with Table 1, below. The personal care formulation may have a pH of about 0.15 to about 2.0, about 0.15 to about 1.8, or about 0.5 to about 1.5, or about 0.65 to about 1.15.

TABLE 1

| | INGREDIENTS | WEIGHT (%) |
|---|---|---|
| 1 | Penetration aids | 15-99 |
| | (e.g., denatured alcohol, witch hazel, urea, | |
| | fatty acids, glycols) | |
| 2 | Alpha hydroxy acids | 7-40 |
| | (e.g., citric acid, glycolic acid, mandelic | |
| | acid, lactic acid, malic acid, tartaric acid) | |
| 3 | Beta hydroxy acids | 1-5 |
| | (e.g., salicylic acid, esters of salicylic | |
| | acid, sodium salicylate, white willow bark | |
| | extract, wintergreen leaves, beta hydroxybutanoic | |
| | acid, tropic acid, trethocanic acid) | |
| 4 | Polyhydroxy acids | up to 22 |
| | (e.g., gluconolactone, galactose, and | |
| | lactobionic acid) | |
| 5 | Humectants | 1-20 |
| | (e.g., polyhydric alcohols such as vegetable | |
| | refined glycerin, butylene glycol, 1,3-propanediol, | |
| | non crystal sorbitol, xylitol or low molecular | |
| | weight polyethylene glycols, polyoxyethylenes, | |
| | polyglutamic acid, saccharide isomerate) | |
| 6 | Soothing agents | up to 5 |
| | (e.g., centella asiatica extract, polygonum | |
| | cuspidatum root extract, scutellaria baicalensis | |
| | root extract, camellia sinensis leaf extract, | |
| | glycyrrhiza glabra root extract, chamomilla | |
| | recutita flower extract, rosmarinus officinalis | |
| | leaf extract, tea leaf extract, poria cocos | |
| | extract, phragmites karka extract) | |
| 7 | Antioxidants, anti-aging agents | up to 5.00 |
| | (e.g., camellia japonica extract, quercetin, | |
| | Morinda citrifolia extract, peony root | |
| | (Paeonia albiflora) extract)) | |
| 8 | Wound healing agents | up to 5.00 |
| | (e.g., triterpenes, triterpenoids, oleanolic acid, | |
| | maslinic acid, asiaticoside) | |
| 9 | Amino acids | up to 10 |
| | (e.g., arginine, taurine, glycine, valine, lysine, | |
| | alanine, cysteine, histidine, leucine, proline, | |
| | serine, tyrosine) | |
| 10 | D.I. Water | q.s .* 100 |

“*” quantum sufficient (as much as is sufficient)

In one embodiment, the present invention provides a method of making the personal care formulations of the invention.

In one embodiment, the present invention provides personal care formulations useful in reducing fine wrinkles and lines, reducing pore size, exfoliating the skin, eliminating acne, toning the skin, enhancing the skin's radiance, and providing softer, smoother skin with a more uniform appearance.

In one embodiment, the present invention provides a method to treat skin aging and improve the skin's appearance, by treating the skin with a personal care formulation of the invention.

In one embodiment, the present invention provides a method to even skin tone, even hyperpigmentation, clear acne, improve the appearance of aging skin and make dull complexions glow, by treating the skin with a personal care formulation of the invention.

In one embodiment, the present invention provides a method to increase the skin's cell turnover rate by stimulating the epidermis to produce fresh skin, bringing new skin cells to the surface, reducing signs of fine lines, wrinkles, dark spots and clearing breakouts by treating the skin with a personal care formulation of the invention.

In one embodiment, the present invention provides a method to treat hyperpigmented and photoaged skin due to sun damage, by treating the skin with a personal care formulation of the invention.

In one embodiment, the present invention provides a method to treat dermatological disorders including dry skin, acne, dandruff, keratosis, age spots, wrinkles and disturbed keratinization, by treating the skin with a personal care formulation of the invention.

In one embodiment, the present invention provides a method for treating the skin, comprising applying to the skin a personal care formulation, wherein the pH of the formulation is between about 0.15 and 2.0, preferably between about 0.15 and about 1.15, more preferably about 0.65; and wherein the personal care formulation comprises the ingredients according to Table 1.

The personal care formulations of the present invention may be formulated as oil-in-water, where oil is in the dispersed phase, and water is the dispersion medium, or water-in-oil, where the reverse is true, emulsions, comprising emulsifiers, gels, liquids, creams, toners, pastes, skin lotions, serums. The personal care formulations of the present invention may be formulated as a solution, suspension, emulsion, paste, gel, cream, sheet mask, lotion, powder, soap, surfactant-containing cleansing, oil, powder foundation, emulsion foundation, wax foundation, or a spray. The personal care formulations of the present invention may be applied with muslin cloths, sponges, brushes, gloves, pads, paper, single-use pads, single use paper masks, cotton and microdermabrasion. The personal care formulations of the present invention may be formulated, packaged and provided in a kit format, comprising one or more of the chemical peel formulations, along with instructions for their use. Such kits may optionally include a means for estimating or measuring the pH of skin before and after treatment with one or more components of the kit. If a means for estimating or measuring the pH of skin before and after treatment is included, it can further require the inclusion of pH testing reagents, indicator (litmus) papers, or a non-invasive, or electronic means. The kit may comprise a post-peel mask for soothing the effect of the chemical peel.

The personal care formulations of the invention may optionally comprise preservative systems, wherein the preservative systems may include, but are not limited to, imidazolidinyl urea, diazolidinyl urea, phenoxyethanol, methylparaben, ethylparaben, and propylparaben.

The personal care formulations of the invention may be used by non-professional subjects such as consumers for at-home treatment, the formulations being capable of providing an improvement of the skin comparable to results obtainable only by professionals using higher concentrations of acid.

The personal care formulations of the invention may be formulated as one step treatment or as multiple step treatment to be used sequentially on the skin of the human subject.

The personal care formulations of the invention does not comprise mineral acids, wherein the mineral acids include, but are not limited to, trichloracetic acid.

The personal care formulations of the invention may optionally comprise metals wherein the metals include, but are not limited to, selenium, zinc, copper, and silicon.

The personal care formulations of the invention may optionally comprise cooling agents, wherein the cooling agents, include, but are not limited to mint oil, cyclic α-keto enamines, aloe vera, chamomile, green tea, peppermint, lemon, eucalyptus and rose.

The personal care formulations of the invention may optionally comprise vitamins and vitamin derivatives, wherein the vitamins may include, but are not limited to, Vitamin A, retinyl acetate, retinyl palmitate, Vitamin C, ascorbic acid, ascorbyl palmitate, magnesium ascorbyl palmitate, magnesium ascorbyl phosphate, Vitamin E, and tocopherol.

The personal care formulations of the invention may optionally comprise botanicals, wherein the botanicals include, but are not limited to, carotenoids, polyphenols, apigenin (flavonoid occurring in numerous herbs, fruits and vegetables), quercetin (flavonol found in onion skin and apple peel), curcumin (from turmeric rhizome), silymarin (extract of flavonolignans from milk thistle), isoflavones, genistein (isoflavone from soybeans), proanthocyanidins (from seeds of grapes), and resveratrol (polyphenol found in grapes, peanuts, fruits, red wine and mulberries).

Additional or alternative humectants that may optionally be present in the personal care formulations include, but are not limited to, glucuronic acid, N-acetyl-glucosamine, hyaluronic acid of different molecular weights, different salts of hyaluronic acid (acetylated hyaluronic acid, sodium acetylated hyaluronate, sodium hyaluronate, potassium hyaluronate).

The personal care formulations of the invention may optionally comprise fatty acids, wherein the fatty acids include, but are not limited to, polyunsaturated omega-3, omega-6 fatty acids, alpha-linolenic acid (ALA), linoleic acid (LA), gamma linolenic (GLA) acid, docosahexanaenoic acid (DHA), eicosapentaenoic acid (EPA), arachidonic acid (AA), cholesterol, ceramides.

The personal care formulations of the invention may optionally comprise antimicrobials, wherein the antimicrobials may include, but are not limited to, benzoyl peroxide, erythromycin, tetracycline, triclosan, azelaic acid, clindamycin, chlorhexidine, tetracycline, neomycin, miconazole, clotrimazole.

The personal care formulations of the invention may optionally comprise oils, wherein the oils include, but are not limited to, borage oil, evening primrose oil, blackcurrant seed oil, hemp oil, baobab oil, neem oil, black cumin oil, coconut oil, sea buckthorn oil, green coffee oil, wheat germ oil, barbary fig oil, argan oil, flaxseed oil.

The personal care formulations of the invention may optionally comprise aesthetic components such as fragrances, pigments, colorants, essential oils, skin sensates, and astringents. Such aesthetic components include, but are not limited to, clove oil, menthol, camphor, eucalyptus oil, eugenol, menthyl lactate, bisabolol, green tea extract.

The personal care formulations of the invention may optionally comprise absorbents, antifoaming agents, antimicrobial agents, binders, biological additives, chelating agents denaturants, external analgesics, steroidal anti-inflammatory drugs, reducing agents, skin bleaching agents, skin protectants, solubilizing agents, solvents, and thickening agents.

The personal care formulations of the invention may optionally comprise skin bleaching agents, wherein the skin bleaching agents include, but are not limited to, hydroquinone, kojic acid, sodium metabisulfite, licorice extract, resorcinol, phenylethyl resorcinol.

The personal care formulations of the invention may optionally comprise peptides, wherein the peptides, known as glycyl-L-histidyl-L-lysine (GHK), bind easily to copper enzymes, forming GHK-Cu.

The personal care formulations of the invention may optionally comprise bioactive compounds naturally produced by marine algae, wherein these bioactive compounds are seaweed crystals, which penetrate the epidermal layer of the skin, and reduce acne inflammation and oil production.

The personal care formulations of the invention may optionally comprise surfactants and/or emulsifying agents, wherein the surfactants and/or emulsifying agents include but are not limited to, ceteareths, ceteths, cetyl alcohol, deceths, dodoxynols, glyceryl palmitate, glyceryl stearate, laneths, myreths, nonoxynols, octoxynols, oleths, PEG-castor oil, poloxamers, poloxamines, polysorbates, ammonium laureth sulfate and sodium laureth sulfate, octoxynol-9 and polysorbate-20.

In some embodiments, the present invention provides a personal care composition comprising: lactic acid; glycolic acid; salicylic acid; and a cosmetically acceptable carrier. In other embodiments, the present invention provides a personal care composition comprising: from about 7 wt. % to about 12 wt. % lactic acid; from about 8 wt. % to about 40 wt. % glycolic acid; from about 1 wt. % to about 8 wt. % salicylic acid; and a cosmetically acceptable carrier.

In other embodiments, the present invention provides personal care compositions comprising: from about 8 wt. % to about 10 wt. % lactic acid. In further embodiments, the present invention provides from about 28 to about 38 wt. % glycolic acid. In some embodiments, the present invention provides a personal care composition comprising: from about 1 wt. % to about 3 wt. % salicylic acid. In certain embodiments, the present invention provides personal care compositions wherein the weight ratio of lactic acid to glycolic acid is from about 1:15 to about 1:20.

In certain embodiments, the cosmetically acceptable carrier comprises a monohydric alcohol. In further embodiments, compositions of the present invention may be substantially free, or free, of trichloroacetic acid.

In some embodiments, the composition has a pH of from about 0.15 to about 2.0, or about 0.15 to about 1.15, or about 0.65.

Still other embodiments provide personal care compositions, wherein the total acid concentration is about 40 wt. % or less. Yet other embodiments provide personal care compositions, wherein the total acid concentration is about 40 wt. %.

In some embodiments, the personal care compositions of the present invention comprise from about 2 wt. % to about 5 wt. % gluconolactone, optionally about 4 wt. % gluconolactone.

In other embodiments, the present invention provides personal care compositions comprising from about 1 wt. % to about 3 wt. % salicylic acid, optionally about 2 wt. % salicylic acid.

In further embodiments, the present invention comprises a cosmetically acceptable carrier comprising from about 15 wt. % to about 50 wt. % of an astringent, optionally about 50 wt. % of an astringent. In some embodiments, the astringent comprises witch hazel. In some embodiments, the personal care composition comprises a cosmetically acceptable carrier comprising from about 5 wt. % to about 18 wt. % of water.

In certain embodiments, the personal care compositions according to present invention comprise a nucleoside, a peptide and a combination thereof. In further embodiments, the nucleoside comprises adenosine. Yet other embodiments of the present invention provide personal care compositions wherein the peptide comprises a dipeptide. Still further embodiments of the present invention provide personal care compositions wherein the peptide comprises carnosine.

In some embodiments, the present invention provides personal care compositions comprising a pH adjusting agent (e.g., sodium hydroxide). In other embodiments, the present invention provides personal care compositions comprising a cosmetically acceptable carrier comprising an ingredient selected from: a fragrance; a surfactant; a particle (e.g., a microbead); a thickening agent; and a combination of two or more thereof.

In some embodiments, the present invention provides personal care compositions comprising a cosmetically acceptable carrier comprising particles. In further embodiments, the present invention provides personal care compositions, wherein the particles comprise gold nanoparticles. In certain embodiments, the present invention provides a personal care composition comprising gold nanoparticles that are functionalized with a peptide. In other embodiments, the present invention provides a personal care composition, wherein the gold nanoparticles are functionalized with a peptide comprising palmitoyl pentapeptide-4 and asiaticoside.

In some embodiments, the present invention provides a personal care composition in a form selected from a body wash; a hand soap; a shower gel; a wipe; a lotion; a cream; an ointment; an antiperspirant; a peel (e.g., a chemical peel); and a deodorant.

Other embodiments of the present invention provide a method for treating, inhibiting or preventing: dyspigmentation of the skin; wrinkles; acne or rosacea; loss of skin elasticity; crow's feet; pigmented lesions (freckles, sun spots, melasma); and/or photo damage to the skin, comprising: applying a personal care composition as described herein to a skin surface of a subject in need thereof.

Further embodiments of the present invention provide a method for: improving photodamage; reducing or preventing fine lines and wrinkles; improving skin tone unevenness; and/or improving mottled pigmentation, comprising: applying a personal care composition as described herein, to a skin surface of a subject in need thereof.

Yet other embodiments provide a method for rejuvenating the skin comprising: applying any one of the personal care compositions described herein to a skin surface of a subject in need thereof, optionally wherein the personal care composition is applied to the skin surface after a dermatological procedure. In some embodiments, the dermatological procedure is selected from: microdermabrasion; ablative laser resurfacing (e.g., with CO₂ or erbium); non-ablative resurfacing (e.g., photo rejuvenation or pulsed dye laser); micro-needling; ultrasound; cryotherapy; radiofrequency; and a combination of two or more thereof.

In some embodiments, the present invention provides a method of increasing the firmness and/or elasticity of the skin of a patient in need thereof, comprising administering to the skin of said patient, an effective amount of a personal care composition as described herein.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

### EXAMPLES

### Example 1

Described below are exemplary compositions of the present invention. The compositions have a pH between 1.0 and 2.0; and can be prepared in accordance with conventional methods known to those skilled in the art.

TABLE 2
Ex. I

| Ingredient | Wt. % |
|---|---|
| Water | 7.800 |
| Ethyl Alcohol | 35.00 |
| Witch Hazel | 15.00 |
| Lactic Acid | 8.00 |
| Glycolic Acid | 30.00 |
| Salicylic Acid | 2.00 |
| Soothing Agents | 0.50 |
| Anti-Aging Agents | 1.70 |

### Example 2

The compositions have a pH of about 3.0-3.5; and can be prepared in accordance with conventional methods known to those skilled in the art.

TABLE 3
Ex. II

| Ingredient | Wt. % |
|---|---|
| Water | 12.71 |
| Alcohol, denatured | 35.00 |
| Witch Hazel | 20.00 |
| Gluconolactone, FCC | 4.00 |
| Lactic acid | 8.00 |
| Glycolic acid | 10.00 |
| Mandelic acid | 6.00 |
| Humectant | 2.00 |
| Salicylic acid | 2.00 |
| Peptides | 0.14 |
| Water, Xanthan gum, | 0.15 |
| Gold, Palmitoyl | |
| pentapeptide-4, | |
| Asiaticoside | |
| NaOH 50% | QS Titrate to |
| | pH 3.0-3.5 |

### Example 3

The compositions have a pH of about 2.0; and can be prepared in accordance with conventional methods known to those skilled in the art.

TABLE 4
Ex. III. 30% glycolic base

| Material | % |
|---|---|
| Demineralized water | 7.500 |
| Alcohol, Denatured | 35.00 |
| Witch Hazel | 15.00 |
| Lactic Acid | 8.00 |
| Glycolic acid | 30.00 |
| Salicylic Acid | 2.00 |
| Anti-Aging Agents | 2.00 |
| NaOH | 0.500 |

### Example 4

The compositions have a pH of about 3.0; and can be prepared in accordance with conventional methods known to those skilled in the art.

TABLE 5
Ex. IV. 20% glycolic base

| Material | % |
|---|---|
| Demineralized water | 17.837 |
| Alcohol, Denatured | 35.00 |
| Witch Hazel | 15.00 |
| Lactic Acid | 8.00 |
| Glycolic acid | 20.00 |
| Salicylic Acid | 2.00 |
| Anti-Aging Agents | 2.00 |
| NaOH | 0.164 |

### Example 5

To assess the effectiveness of chitosan as a thickening agent for chemical peels, prototypes are prepared that consist of glycolic acid in water. Glycolic acid is a relatively small acid that can readily enter into the skin; therefore slowing its rate of release would be of high interest. Additionally, no endogenous glycolic acid exists in the skin (in contrast to other common peeling acids like lactic acid), allowing its topically delivered levels in the skin to be conveniently measured using HPLC. Chitosan is incorporated into aqueous solutions of glycolic acid and the efficacy is measured using a variety of skin tests, described below. The delivery of glycolic acid into and through porcine ear skin explants are measured in a series of in vitro penetration tests (IVPTs). Prototypes are then tested on the arms of several human volunteers. The irritation level of the participants is recorded, and for one participant the irritation in the upper layers of the skin is visually assessed using a Vivascope reflectance confocal microscope, where inflammatory cells are visible as bright white dots in the epidermal region.

The specific chitosan used in the study is KiOsmetine-CsH, supplied by KitoZyme s.a. Addition of this material to simple chemical peels imparts the formulas with a light, golden-brown-like color.

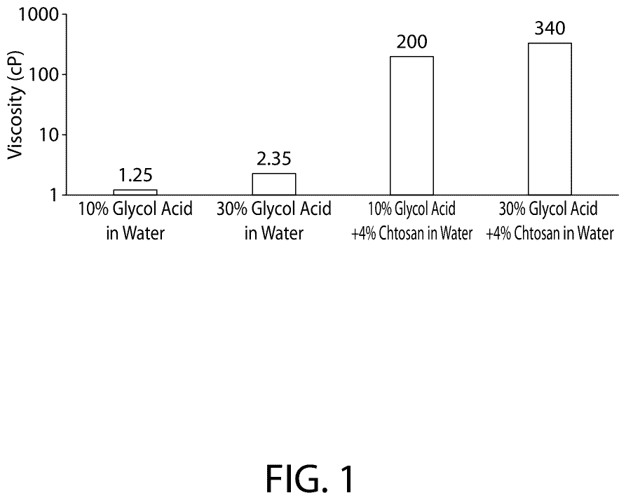

Although the viscosity increasing properties are clearly visible with the naked eyes, the actual viscosity of samples are measured with a rheometer. The samples are simple aqueous solutions of either 10% or 30% glycolic acid, with or without 4% chitosan. Viscosity measurements are acquired with a DIN concentric cylinder geometry with an ARES Rheometer, using a shear rate range of 10-100/sec. FIG. 1 depicts a graph of the viscosity results, where it can be seen that the addition of 4% chitosan causes a large increase in measured viscosity, with the values being roughly 100 times larger than the counterparts that do not contain chitosan. Increasing the glycolic acid from 10% to 30% causes a small increase in viscosity, although the main increase in viscosity is caused by the inclusion of the chitosan.

IVPT experiments are conducted on a DHC-6T Franz diffusion cell from Logan instruments (Somerset, New Jersey). Studies are conducted using porcine ear skin acquired from Animal Technologies (Tyler, Texas). To analyze the levels of glycolic acid that penetrate into or through the skin, a modified derivatization protocol was developed based on published literature (Pourasghar, Marcel, et al. Journal of Pharmaceutical Analysis 9.2 (2019):100-107). The derivatized glycolic acid is analyzed on an HPLC system (either an Agilent 1200 quaternary pump system or an Agilent 1260 quaternary pump system). Samples are run using a C18 reverse phase column, with a gradient mobile phase that starts at 90/10 water/acetonitrile, then increases the organic phase to 100% acetonitrile, and then reverts back to the starting composition. The derivatized analyte is measured at 254 nm. The flow rate is 1 mL/min with an injection volume of L.

For the experimental setup, porcine ear skin is placed at the top of each individual Franz cell (the receiver compartment). The Franz cell is filled with DI water that has been heated to 32 C. Then, 20 μL of either sample is applied to the surface of the skin and rubbed on for 10 seconds to ensure even application. The topmost chamber is clamped on and the sample is left to penetrate into the skin over a defined time period (1, 3, 6, and 24 hours). Afterwards, the skin itself and sample from the receiving fluid are both collected, and the levels of glycolic acid in each region are analyzed via HPLC.

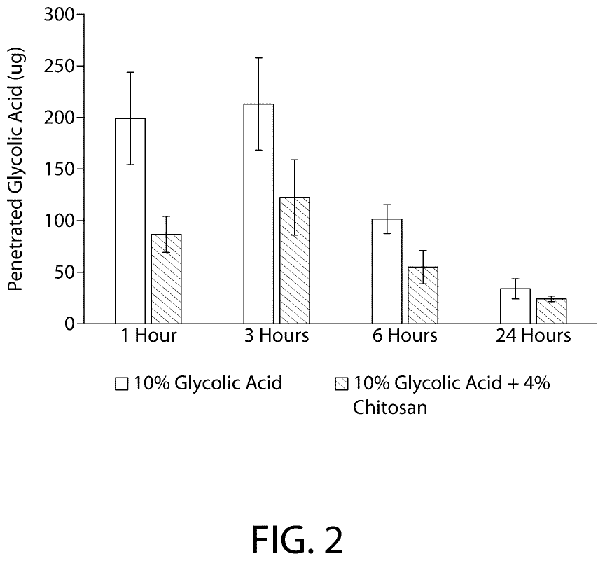

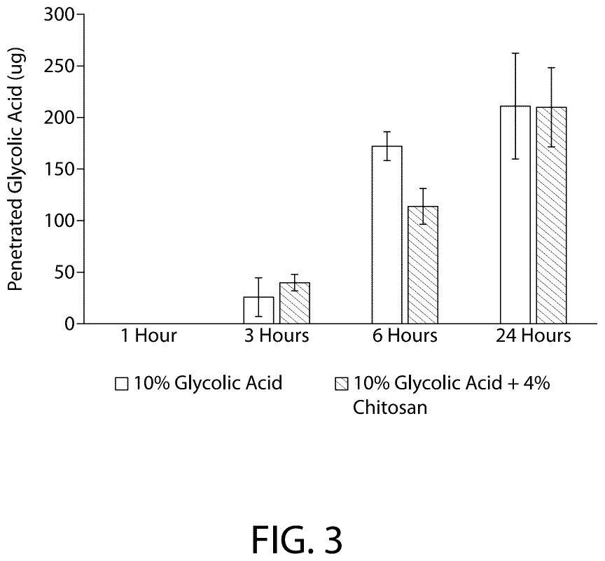

The amount of glycolic acid within the skin, and the amount of glycolic acid penetrating through the skin and into the receiver fluid are listed in FIGS. 2 and 3, respectively. The data is listed below in Tables 6 and 7, respectively. Clearly, the inclusion of chitosan in the formulas makes a clear difference in the level and overall kinetics of how glycolic acid is delivered into the skin. Within the skin itself, the samples without chitosan (10% glycolic acid in water) have significantly higher levels of glycolic acid after 1, 3 and 6 hours than the samples with both glycolic acid and 4% chitosan (p<0.05). After 24 hours, both samples have delivered similarly low levels of glycolic acid into the skin, with no statistically significant difference (p>0.10).

TABLE 6

| | | | |
|---|---|---|---|
| Glycolic Acid in | Sample without | Sample with | |
| the Skin (μg) | Chitosan | 4% Chitosan | T Test |

| | | | | |
|---|---|---|---|---|
| 1 | Hour | 199.04 ± 44.86 | 86.36 ± 17.43 | 0.0017 |
| 3 | Hours | 213.34 ± 44.58 | 122.68 ± 36.36 | 0.020 |
| 6 | Hours | 101.01 ± 13.42 | 54.87 ± 15.75 | 0.0043 |
| 24 | Hours | 33.62 ± 9.85 | 23.83 ± 2.48 | 0.10 |

TABLE 7

| Glycolic Acid | | | | |
|---|---|---|---|---|
| Through the | | | | |
| Skin/Into Receiver | | Sample without | Sample with | |
| Fluid (μg) | | Chitosan | 4% Chitosan | T Test |
| 1 | Hour | n.d | n.d. | n/a |
| 3 | Hours | 26.44 ± 18.07 | 39.4 ± 8.22 | 0.24 |
| 6 | Hours | 172.07 ± 14.36 | 113.29 ± 17.95 | 0.0022 |
| 24 | Hours | 210.80 ± 50.73 | 209.83 ± 38.30 | 0.98 |

A somewhat similar pattern is visible in the level of glycolic acid delivered through the skin and into the receiver fluid (FIG. 3). No glycolic acid is detected after 1 hour as the time period is not long enough for the glycolic acid to penetrate through the skin. At 3 hours, both samples have delivered very small levels of glycolic acid into the skin, with the difference between the two not being statistically significant (p>0.10). At six hours, the sample without chitosan has delivered a larger amount of glycolic acid into the receiver fluid than the sample with chitosan, this difference being statistically significant (p<0.05). Finally, after 24 hours the level of glycolic acid in the receiver fluid is nearly identical between the two samples, with no statistically significant difference (p>0.10).

From the data present in FIGS. 2 and 3, it can be seen that the addition of chitosan serves to provide a more sustained rate of release of glycolic acid into the skin, rather than alter and/or lower the total amount of glycolic acid that is delivered. Samples with and without chitosan have nearly identical levels of glycolic acid delivered into and through the skin after 24 hours; however, they achieve this level with differing routes. Samples without chitosan have high levels of glycolic acid that rapidly enter the skin after 1 and 3 hours. At 6 hours much larger levels of glycolic acid have consequently cleared the skin and entered the receiver fluid. In contrast, samples with glycolic acid and chitosan have lower levels of glycolic acid that have entered the skin at 1 and 3 hours, indicative of a slower rate of release due to the thickening properties of chitosan.

### Example 6

To further explore the kinetics of the glycolic acid delivery into the skin, a full breakdown of the glycolic acid is explored at a single time point of 3 hours. To achieve this, the initial procedure from above is followed with two additional steps. First, any residual glycolic acid on the skin surface is collected using cotton swabs. Second, the level of glycolic acid within the first 20 tape strips of the skin is analyzed and measured. (In the previous experiments the first 20 tape strips are discarded). Without being bound by theory, by adding the measurements obtained at these two regions, the glycolic acid level should be able to be analyzed at any conceivable region at which it might be present.

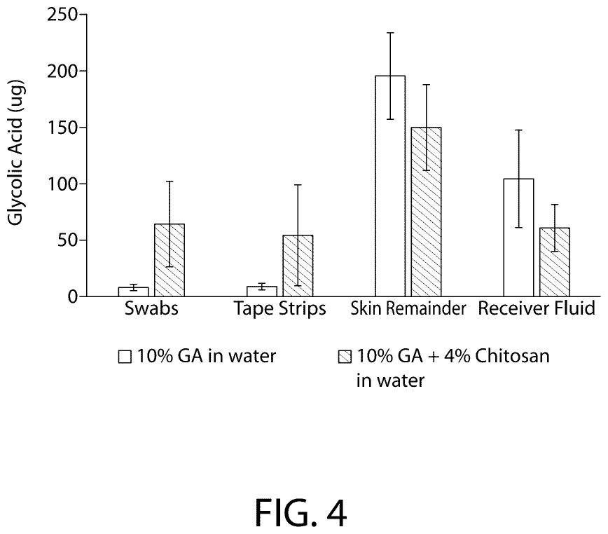

The results of this full breakdown are shown in FIG. 4, and the data is shown in Table 8, below. Similar trends emerge as to what is observed in FIGS. 2 and 3. The sample with 10% glycolic acid in water (without chitosan) has higher levels of glycolic acid penetrating into the skin and through the skin into the receiver fluid, with minimal levels present in the cotton swabs or tape strips. Conversely, the sample containing 10% glycolic acid and 4% chitosan has lower levels of glycolic acid in both the skin and the receiver fluid; however, it has significantly higher levels of glycolic acid in both the tape strips and cotton swabs (p<0.10 for both tape strips and cotton swabs). This indicates that for time points when the chitosan-containing samples have less glycolic acid in the skin and the receiver fluid, it is because there is a reservoir of glycolic acid remaining in the uppermost tape strip regions and on the surface of the skin itself, as manifested in the collected cotton swabs. This is not the case for the samples without chitosan, as they have virtually no remaining glycolic acid in either the swab or tape strips regions after 3 hours. This indicates that all or almost all of the glycolic acid has entered into deep regions of the skin by this point.

TABLE 8

| Glycolic | 10% Glycolic | 10% Glycolic | |
|---|---|---|---|
| Acid Level | Acid | Acid + 4% | |
| (μg) | in Water | Chitosan in Water | T Test |
| Cotton Swabs | 8.94 ± 2.03 | 64.79 ± 37.96 | 0.026 |
| Tape Strips | 9.57 ± 3.25 | 55.05 ± 44.36 | 0.086 |
| Skin | 195.63 ± 38.18 | 150.30 ± 38.24 | 0.14 |
| Receiver Fluid | 104.63 ± 43.10 | 61.46 ± 20.56 | 0.12 |

### Example 7

With the above penetration data, it is hypothesized that the addition of chitosan would lead to reduced irritation in a chemical peel prototype. To test this, a 30% solution of glycolic acid in water is prepared, along with an aqueous solution of 30% glycolic acid and 4% chitosan. These products are applied to the volar forearms of volunteers, who then assessed the area for any irritation. 100 μL of each product is applied to a 5 cm by 5 cm square region, and then rubbed on with a gloved finger for 10 seconds. Of the five volunteers tested, one was removed as significant portions of the peel dripped off during application, leading to invalid results.

The four volunteers all experienced a more intense irritation on the arms treated with the product with no chitosan. The arm treated with the chitosan-containing product has much less/no irritation. This indicates that the sustained release properties afforded by the chitosan ultimately results in a significant reduction in irritation during peel application.

In summary, we have demonstrated the use of chitosan as a sustained release agent with applications in chemical peels. By raising the viscosity of a chemical peel formulation, chitosan can allow the acid of interest (in this work glycolic acid) to enter into the skin in a slower and more sustained manner, as demonstrated by examining several key time points. However, at a longer time point, formulas with and without glycolic acid deliver the same amount of active into the skin. This indicates that chitosan simply provides a slower, more sustained route of release, without negatively affecting the total amount of active delivered.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

What is claimed is:

1. A personal care composition comprising:
from about 7 wt. % to about 12 wt. % lactic acid;
from about 8 wt. % to about 40 wt. % glycolic acid;
from about 1 wt. % to about 8 wt. % salicylic acid;
a naturally derived thickening agent; and
a cosmetically acceptable carrier;
wherein the personal care composition is substantially free, or free of trichloroacetic acid (TCA).

2. The personal care composition according to claim 1, wherein the naturally derived thickening agent comprises chitosan.

3. The personal care composition according to claim 1, wherein the total acid concentration is about 50 wt. % or less by weight of the composition.

4. The personal care composition according to claim 1, comprising lactic acid in an amount of from about 6 wt. % to about 10 wt. %, preferably about 8 wt. %, by weight of the composition.

5. The personal care composition according to claim 1, comprising glycloic acid in an amount of from about 10 wt. % to about 35 wt. %, preferably about 20 wt. % to about 30 wt. %, more preferably about 30 wt. %, by weight of the composition.

6. The personal care composition according to claim 1, comprising salicylic acid in an amount of from about 1 wt. % to about 2 wt. %, preferably about 2 wt. %, by weight of the composition.

7. The personal care composition according to claim 1, wherein the weight ratio of lactic acid to glycolic acid is from about 1:3 to about 1:4, or about 1:3.75.

8. The personal care composition according to claim 1, wherein the cosmetically acceptable carrier comprises an ingredient selected from: water, an astringent, a monohydric alcohol, a fragrance, a surfactant, a microbead, a further thickening agent, and a combination of two or more thereof.

9. The personal care composition according to claim 8, wherein the cosmetically acceptable carrier comprises from about 5 wt. % to about 10 wt. % of water.

10. The personal care composition according to claim 8, wherein the cosmetically acceptable carrier comprises an astringent in an amount of from about 10 wt. % to about 60 wt. %, by weight of the composition;
wherein the astringent comprises ethanol, witch hazel, or a combination thereof.

11. The personal care composition according to claim 10, wherein the witch hazel is present in an amount of about 10 wt. % to about 20 wt. %, or about 15 wt. % by weight of the composition.

12. The personal care composition according to claim 1, wherein the personal care composition further comprises bakuchiol.

13. The personal care composition according to claim 1, wherein the composition has a pH of from about 1.0 to about 2.0.

14. The personal care composition according to claim 1, wherein the composition further comprises a nucleoside, a peptide and a combination thereof;
wherein the peptide comprises palmitoyl pentapeptide-4 and asiaticoside.

15. The personal care composition according to claim 1, wherein the composition is in a form selected from a body wash, a hand soap, a shower gel, a wipe, a lotion, a cream, an ointment, an antiperspirant, a chemical peel, and a deodorant.

16. A personal care composition comprising:
from about 7 wt. % to about 12 wt. % lactic acid;
from about 8 wt. % to about 35 wt. % glycolic acid;
from about 2 wt. % to about 8 wt. % salicylic acid;
bakuchiol; and
a cosmetically acceptable carrier;
wherein the personal care composition is substantially free of trichloroacetic acid (TCA).

17. A method for treating, inhibiting or preventing:
dyspigmentation of the skin;
wrinkles;
acne or rosacea;
loss of skin elasticity;
crow's feet;
pigmented lesions including freckles, sun spots, melasma; and/or
photo damage to the skin,
comprising:
applying a personal care composition according to claim 1 to a skin surface of a subject in need thereof.

18. A method for:
improving photodamage;
reducing or preventing fine lines and wrinkles;
improving skin tone unevenness; and/or
improving mottled pigmentation,
comprising:
applying a personal care composition according to claim 1, to a skin surface of a subject in need thereof.

19. A method for rejuvenating the skin, comprising: applying the personal care composition of claim 1 to a skin surface of a subject in need thereof,
wherein the personal care composition is applied to the skin surface after a dermatological procedure,
wherein the dermatological procedure is selected from: microdermabrasion; ablative laser resurfacing with CO₂; ablative laser resurfacing with erbium; non-ablative resurfacing, such as photo rejuvenation or pulsed dye laser; micro-needling; ultrasound; cryotherapy; radiofrequency; and a combination of two or more thereof.

20. A method of increasing the firmness and/or elasticity of the skin of a patient in need thereof, comprising administering to the skin of said patient, an effective amount of a personal care composition according to claim 1.
